(19) United States
(12) Patent Application Publication
(10) Pub. No.: US 2008/0026039 A1
(43) Pub. Date: Jan. 31, 2008

(54) Benzoyl peroxide pads and formulations therefor

(75) Inventor: Karl F. Popp, Schodack Landing, NY (US)

Correspondence Address: NATH & ASSOCIATES, 112 South West Street, Alexandria, VA, 22314, US

(73) Assignee: STIEFEL LABORATORIES, INC., Coral Gables, FL (US)

(21) Appl. No.: 11/492,773

(22) Filed: Jul. 26, 2006

(51) Int. Cl. A61K 9/70; A61K 31/192

(52) U.S. Cl. 424/443; 514/568

(57) ABSTRACT

Pharmaceutical delivery systems suitable for topical administration including benzoyl peroxide. In a particular aspect, these pharmaceutical delivery systems comprise at least one pad and a drug composition comprising benzoyl peroxide absorbed onto the pad. These compositions are used for topical medical applications, particularly to treat various skin disorders.

### FIELD OF THE INVENTION

The present subject matter relates generally to drug delivery systems suitable for topical administration of benzoyl peroxide. In a particular aspect, these pharmaceutical delivery systems comprise at least one pad and a drug composition comprising benzoyl peroxide absorbed onto the pad. These compositions are used for topical medical applications, particularly to treat various skin disorders.

### BACKGROUND OF THE INVENTION

Acne is a condition of the human skin characterized by an excess flow of sebum, or skin oil, from the sebaceous glands located in the pilosebaceous apparatus. Sebum reaches the skin surface through the duct of the hair follicle. The presence of excessive amounts of sebum in the duct and on the skin acts to block or stagnate the continuous flow of sebum from the follicular duct, thus producing a thickening and a solidification of the sebum to form a solid plug known as a comedone. When this process occurs, hyperkeratinization of the follicular opening is stimulated, thus completely closing the duct. The usual results are papules, pustules, or cysts, often contaminated with bacteria which cause secondary infections. Acne is particularly characterized by the presence of comedones, inflammatory papules, pustules, or cysts. The effect of acne ranges from slight skin irritation and pitting to disfiguring scars.

Many topical therapeutic agents are employed in the treatment of acne and seborrhea to prevent the blocking of the follicular duct, to reopen the duct once it has become blocked, to act against the infecting bacteria or the thickened sebum, or to provide combinations of each of these actions. The horny outer layer of the skin, which is known as the stratum corneum, is formed of dead cells composed largely of keratin. Therapeutic agents which act to prevent the blocking of the follicular duct by promoting the removal or sloughing off of excess keratin are known as keratolytic agents.

Benzoyl peroxide has been used as a very effective keratolytic and antibacterial agent in the treatment of acne. The topical application of benzoyl peroxide for skin lesion therapy is well known. For example, U.S. Pat. Nos. 5,445,823, 5,545,407, and 5,932,228 disclose compositions for treating acne and other skin lesions and also to methods of treatment utilizing these compositions. These compositions and methods of treatment employ benzoyl peroxide, a compound for reducing the skin irritation associated with benzoyl peroxide, and a topical carrier.

Similarly, U.S. patent application Publication No. 2004/0101566 discloses nanoparticulate compositions comprising benzoyl peroxide. The benzoyl peroxide particles of the composition have an effective average particle size of less than about 2 microns. The nanoparticulate benzoyl peroxide is used in methods of treating cutaneous disorders.

Likewise, U.S. patent application Publication No. 2005/0255133 discloses a topical composition for treatment of skin disorders such as acne. The composition contains benzoyl peroxide in an amount from between 0.5 and 20% by weight. There is also provided a water miscible solvent in an amount between 10% and 95% by weight for solubilizing the benzoyl peroxide. Finally, a water miscible or water dispersible surfactant is present in an amount between 0.5 and 95% by weight.

In addition, U.S patent application Publication No. 2003/0077301 discloses a topical pharmaceutical composition for the treatment of inflammatory dermatoses, including acne vulgaris, together with a method of use of the topical composition. The composition and method involve the topical use of an active agent effective in the treatment of inflammatory dermatoses plus a permeation-enhancing base that gives the composition a pH of about 8 to about 13, preferably about 8 to 11.5, and most preferably about 8.5 to 10.5. The active agent can be benzoyl peroxide.

Benzoyl peroxide is also found in a variety of over-the-counter and prescription acne products which take the form of lotions, creams or gels. Tubes and bottles of acne medicines are the most common ways of packaging the compositions containing the anti-acne medications, including benzoyl peroxide. However, tubes and bottles are inconvenient for patients to carry to school, camp, office, etc. Consequently, over the last decade a new method of delivering many anti-acne agents has evolved based on incorporating active anti-acne ingredients into small cloth towelettes called wipes or pledgettes. These pledgettes can then be packaged in a sealed pouch that can be conveniently opened at the time of use. Additionally, since only one dose is opened at a time, several patients can “share” a box of such pledgettes without exposure to one another's germs, dirt, etc.

Topical antibiotics are another popular prescription treatment for acne. Today, wipes or pledgettes containing topical antibiotics such as clindamycin and erythromycin are widely used for their convenience, as well as their safety and efficacy.

Attempts to incorporate benzoyl peroxide into wipes or pledgettes have also been made. For example, U.S. Pat. No. 6,740,330 discloses an article for use in the treatment of acne vulgaris comprising a cloth pledgette impregnated with a composition comprising benzoyl peroxide and an amount of acetone sufficient to solubilize the benzoyl peroxide. In a preferred embodiment, the article is packaged in an individual pouch.

Meanwhile, U.S. Pat. Nos. 5,242,433, 5,254,109, 5,368,581, 5,417,674, 5,460,620, 5,470,323, and 5,562,642 all disclose a system and method for applying a plurality, preferably two, dermatological agents to the skin from a single dispensing and applicator system comprising a plurality of compartmentalized applicator pads which may be exposed and sequentially or simultaneously applied to the skin area to be treated. One pad of the acne treatment system will preferably comprise an effective amount of an organic peroxide, such as benzoyl peroxide.

Additionally, U.S. Pat. Nos. 5,538,732 and 6,001,380 disclose a method for applying a plurality of dermatological agents to the skin from a single dispensing and applicator sheet comprising a plurality of discrete areas. The discrete areas comprise at least two dermatological agents which are simultaneously released from the sheet and applied to the afflicted skin area when the sheet is rubbed over wet skin.

Further, U.S. patent application Publication Nos. 2005/0025817, 2005/0100585, and 2005/0232978 disclose a delivery system comprising a pad and an emulsion composition thereon. The emulsion composition comprises an insoluble dermatologically active ingredient with a viscosity that permits substantially uniform absorption of the composition onto the pad. The pad is then packaged in a sealed container. Benzoyl peroxide is a preferred active ingredient in the disclosed delivery system. The benzoyl peroxide is incorporated into the emulsion composition prior to being retained by the pad.

However, the prior art delivery systems for benzoyl peroxide still contain drawbacks. Benzoyl peroxide is generally not soluble in water, and thus has been marketed in the form of suspensions in creams or lotions. Such creams or lotions are not readily applicable to a user's afflicted skin through the use of pads since creams or lotions do not readily adhere to the pads. In addition, creams or lotions traditionally have a higher viscosity, which creates difficulties in applying the cream or lotion to the skin in an even manner. Low viscosity suspensions of benzoyl peroxide also have problems with the efficacy of the treatment when attempts to apply the low viscosity suspensions with pads are made.

Accordingly, there remains a need in the art for topical compositions containing benzoyl peroxide for treating a dermatological disorder that are capable of being absorbed onto a pad for ease of administration to the skin of a user, and in which the topical compositions are highly efficacious in treating the dermatological disorder. The present subject matter addresses these needs.

### SUMMARY OF THE INVENTION

The present subject matter relates generally to a topical drug delivery system comprising therapeutically effective levels of benzoyl peroxide that exhibits excellent stability characteristics and methods of using the same to treat various skin disorders.

In this regard, a preferred embodiment of the present subject matter relates to a topical drug delivery system comprising:

at least one pad;

a container; and

a drug composition comprising about 2.5-13% by weight benzoyl peroxide in a suspension comprising about 80-90% by weight water and about 0.1-1.5% by weight of a surfactant system;

wherein said drug composition is absorbed onto said pad, and said container holds said pad with said drug composition.

In a preferred embodiment, the surfactant system comprises about 50-95% by weight of at least one nonionic surfactant and about 5-50% by weight of at least one anionic surfactant.

Another preferred embodiment of the present subject matter relates to a topical drug delivery system comprising:

at least one pad; and

a drug composition comprising about 2.5-13% by weight benzoyl peroxide in a suspension comprising about 80-90% by weight water and about 0.1-1.5% by weight of a surfactant system;

wherein said drug composition is absorbed onto said pad.

Yet another preferred embodiment of the present subject matter relates to a topical drug delivery system comprising:

at least one pad;

a container; and

a drug composition comprising about 2.5-13% by weight benzoyl peroxide in a suspension comprising about 80-90% by weight water, about 0.1-1.5% by weight of a surfactant system, about 0.2-0.4% by weight of a gelling agent, and about 2-6% by weight of a moisturizer;

wherein said drug composition is absorbed onto said pad, and said container holds said pad with said drug composition.

Still another preferred embodiment of the present subject matter is directed to a method for treating acne comprising administering to a patient in need thereof a drug composition comprising about 2.5-13% by weight benzoyl peroxide in a suspension comprising about 80-90% by weight water and about 0.1-1.5% by weight of a surfactant system; wherein said drug composition is administered to said patient using a pad on which said drug composition is absorbed.

Still yet another preferred embodiment of the present subject matter relates to a method for treating acne comprising administering to a patient in need thereof a drug composition comprising about 2.5-13% by weight benzoyl peroxide in a suspension comprising about 80-90% by weight water, about 0.1-1.5% by weight of a surfactant system, about 0.2-0.4% by weight of a gelling agent, and about 2-6% by weight of a moisturizer; wherein said drug composition is administered to the patient using a pad on which said drug composition is absorbed.

### DETAILED DESCRIPTION OF THE INVENTION

### DEFINITIONS

As used herein, “absorb”, “absorbs” and “absorbed” are used interchangeably and refer to the relationship between the drug composition and the pad of the present subject matter. In particular, “absorb” refers to the drug composition being removably entrained within the pores of the pad, as well as on the surface of the pad. The drug composition is at least partially absorbed onto and into the pad. The term “absorb” also means that the drug composition may partially adsorb onto the pad. When the drug composition is absorbed into the pad, the active agent becomes entrapped within the pad and is released from the pad when the composition is transferred from the pad to a user's skin.

As used herein, the term “acne” means a common inflammatory disease of the pilosebaceous glands characterized by comedones, papules, pustules, inflamed nodules, superficial pus-filled cysts, and (in extreme cases) canalizing and deep, inflamed, sometimes purulent sacs. Types of acne within the scope of the present subject matter include acne vulgaris or topical acne. “Acne” is caused by an interaction among hormones, keratin, sebum, and bacteria. One common bacterial causative agent is Propionibacterium acnes.

As used herein, “derivative” or “derivatives” refers to derivative(s) of the active compound(s) which possess the same pharmacological activity as the active compound(s) and which are neither biologically nor otherwise undesirable. Derivatives of the active compounds include, without limitation, polymorphs, solvates, salts, N-oxides, hydrates, dehydrates, crystalline forms, anhydrous forms, amorphous forms, and mixtures thereof.

As used herein, an “extended period of time” refers to the shelf life of the presently preferred compositions, including time spent on the shelf at a pharmacy as well as the entire time period after sale of the composition during which the composition remains effective for the indicated use.

As used herein, a “patient” refers to an entity to whom the preferred drug compositions are being administered. Non-limiting examples of a patient in this regard include a mammal, an animal, and a human being. Preferably, the patient is a human being.

As used herein, “pharmaceutically acceptable salts” or “salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained. Preferred salts include acetate, butyrate, hemisuccinate and phosphate.

As used herein, the term “sensitivity” refers to the degree of skin irritation or skin inflammation, as exemplified by parameters in suitable assays for measuring sensitivity, inflammation, irritation, and the like. One such assay is the Jordan-King assay.

As used herein, “storage stable” refers to the ability of the present compositions to have a long shelf life, including time spent on the shelf at a pharmacy as well as the entire time period after sale of the composition, during which time the composition maintains its effectiveness and pharmaceutically acceptable appearance. Accordingly, the present compositions are stable in that they exhibit a minimum amount of degradation during an extended period of storage.

As used herein, “suspension” refers to the relationship between the active agent and the liquid components of the drug composition. The active agent is preferably suspended within the drug composition, meaning that microscopic or nanoscopic particles of the active agent are dispersed within the liquid medium of the drug composition. The active particles are supported by the buoyancy of the liquid medium. The suspension of particles within the liquid medium enables the liquid medium to transport the particles into the pads, where the particles become entrained or entrapped within the voids of the pad.

Other terms as used herein are meant to be defined by their well-known meanings in the art.

### Topical Pharmaceutical Delivery Systems

The present subject matter is directed to a topical drug delivery system comprising:

at least one pad;

a container; and

a drug composition comprising about 2.5-13% by weight benzoyl peroxide in a suspension comprising about 80-90% by weight water and about 0.1-1.5% by weight of a surfactant system;

wherein said drug composition is absorbed onto said pad, and said container holds said pad with said drug composition.

The preferred drug compositions described herein are unique in that they exhibit remarkable stability. Since benzoyl peroxide is well-known for its oxidation properties, it is often difficult to formulate benzoyl peroxide containing compositions that exhibit long term stability. In this regard, the preferred compositions exhibit excellent stability for an extended period of time under normal storage conditions, particularly with respect to the active ingredient present in conjunction with the other components of the drug composition. The excellent storage stability of the preferred compositions solves long felt difficulties in formulating benzoyl peroxide compositions due to the extreme oxidative nature of benzoyl peroxide.

Since the present drug compositions have an increased stability and are absorbable on the pads of the present delivery systems, they provide unexpected advantages over the prior art compositions. For example, the increased storage stability permits the presently preferred compositions to be manufactured in greater quantities without fear that the compositions produced will be wasted. Further, the enhanced stability and absorbability on the pads provides the presently preferred drug compositions with an enhanced effect in treating acne and other skin disorders treatable with benzoyl peroxide over the previously known compositions, which are not generally absorbable on pads.

The enhanced stability of the preferred drug compositions also provides greater control over degradates associated with benzoyl peroxide. As indicated above, benzoyl peroxide usually has very oxidative properties, often leading to the rapid degradation of compositions containing benzoyl peroxide and the formation of many degradates. The enhanced stability of the preferred drug compositions provides greater control over degradate formation when compared with known benzoyl peroxide delivery systems. In this regard, the present compositions are preferably able to maintain a purity of at least 90% and a concentration of degradation product(s) less than about 10% of the starting concentration of the benzoyl peroxide, as well as of the other essential ingredients in particularly preferred embodiments.

As indicated above, the presently preferred drug compositions are suspensions, with the active agent suspended in a liquid medium. The surfactant system helps to incorporate the active particles into the liquid medium. The use of a suspension for absorbing the drug composition into and onto the pads provides a simpler composition over the benzoyl peroxide emulsions of the prior art. The preferred drug compositions are simpler over the emulsions because, as non-emulsions, the preferred drug compositions do not require two phases (a hydrophilic phase and a lipophilic phase) which are combined through the presence of an emulsifier. The preferred drug compositions, on the other hand, use the surfactant system aid in suspending the active particles in the liquid medium, and therefore do not require two different phases to be combined.

The selection of certain components and amounts thereof in the presently preferred drug compositions, as well as the preparation of compositions having a specific viscosity in the form of dispersions or suspensions, conveys these unique stability and absorbability characteristics to the presently preferred drug compositions. In particular, the presence of a surfactant system, as well as a moisturizer, with the recited weight ratios of nonionic and anionic surfactants enhances the stability of the presently preferred compositions, and aids in rendering these compositions well absorbable onto the pads for ease of administration to the patient.

The present compositions are introduced to the pad as a suspension. The benzoyl peroxide present in the drug composition is pharmaceutical grade. The benzoyl peroxide component of the present compositions is generally present at an amount of between about 2.5% to about 13% by weight of the total composition of benzoyl peroxide. In a preferred embodiment, the compositions contain from about 4% to about 8% by weight of the total composition of benzoyl peroxide. In a particularly preferred embodiment, the present compositions contain about 4% or about 8% by weight of benzoyl peroxide. The present compositions are unique in that they can be produced having a standard deviation of benzoyl peroxide present within ±0.07.

Surfactant System

The present drug compositions can additionally preferably contain a surfactant system. Benzoyl peroxide is relatively insoluble in water. Accordingly, the surfactant system used herein is uniquely selected to provide the benzoyl peroxide with the ability to be incorporated into, for example, a water component of the present compositions to produce an overall drug composition that is able to be absorbed onto a pad drug delivery system without the benzoyl peroxide and water forming an emulsion.

In this regard, preferred surfactant systems herein may be present in the drug composition at an amount of about 0.1% to about 1.5% by weight of the total composition. In a particularly preferred embodiment, the drug compositions may contain from about 0.4% to about 1% by weight of the surfactant system. In a most preferred embodiment, the drug compositions may contain about 0.45% by weight of the surfactant system.

The surfactant system preferably comprises a combination of a plurality of surfactants. In preferred embodiments in this regard, the surfactant system comprises at least one nonionic surfactant and at least one anionic surfactant.

In one embodiment in this regard, the surfactant system comprises at least one nonionic surfactant in an amount of about 50-95% by weight of the surfactant system and at least one anionic surfactant in an amount of about 5-50% by weight of the surfactant system. In a preferred embodiment, the surfactant system comprises at least one nonionic surfactant in an amount of about 75-85% by weight of the surfactant system and at least one anionic surfactant present in an amount of about 15-25% by weight of the surfactant system. In a particularly preferred embodiment, the surfactant system comprises at least one nonionic surfactant present in an amount of about 67% by weight of the surfactant system and at least one anionic surfactant present in an amount of about 33% by weight of the surfactant system.

Accordingly, the surfactant system preferably comprises at least one nonionic surfactant. Non-limiting examples of nonionic surfactants useful in the present surfactant systems in this regard include alkanolamides, amine oxides, esterified carboxylic acids, ethoxylated alcohols, poloxamers, and mixtures thereof. In a preferred embodiment, the at least one nonionic surfactant of the surfactant system is a poloxamer.

One of skill in the art will recognize that poloxamers useful herein include a nonionic polyoxyethylene-polyoxypropylene block co-polymer. Non-limiting examples of commercially available poloxamers usable in the present drug compositions are the Pluronict) line of products available from BASF Corporation.

The surfactant system of the present drug compositions may also preferably comprise at least one anionic surfactant. Non-limiting examples of anionic surfactants useful in this regard include carboxylates, amino acid derivatives, alkyl sulphates, alkyl ether sulfates, sulphonates, isethionates, taurates, sulfosuccinates, alkyl sulfoacetates, phosphates, alkyl phosphates, and mixtures thereof. In a preferred embodiment, the at least one anionic surfactant of the surfactant system is disodium lauryl sulfosuccinate.

In a particularly preferred embodiment, the nonionic surfactant of the surfactant system is a poloxamer, while the anionic surfactant is disodium lauryl sulfosuccinate. In a further particularly preferred embodiment, the poloxamer is preferably present in the surfactant system in an amount of 0.2% by weight of the drug composition, while disodium lauryl sulfosuccinate is present in an amount of 0.1% by weight of the drug composition.

This combination of nonionic surfactant and anionic surfactant may act synergistically to allow optimal delivery of the benzoyl peroxide on the skin of the user. Moreover, by virtue of the specific formulations enumerated herein, the release and/or absorption of the active benzoyl peroxide from the preferred compositions may be attained slowly and gradually when the composition is topically applied to the skin, if desired, which can make it very pleasing for use by a patient.

Water

The present drug compositions may also contain water as an aqueous carrier. The water is present as a carrier for the drug compositions, as well as a solvent for solubilizing and/or suspending the benzoyl peroxide in conjunction with the surfactant system. The water is preferably present in the drug compositions in an amount of about 80% to about 90% by weight of the drug composition. In a particularly preferred embodiment, the water is present in an amount of about 88% by weight of the drug composition.

The preferred compositions herein are preferably formed as a solution, or a dispersion in which the surfactant system aids in solubilization of the benzoyl peroxide. The optimization of the drug composition viscosity is particularly important to the storage stability of the present compositions. Often, the viscosity of solutions will increase when the compositions are stored for long periods of time. The present drug compositions, though, exhibit excellent stability with respect to the viscosity of the drug compositions, even after the compositions have been stored for a desired period of time.

Moisturizer

In a preferred embodiment, the present drug compositions may further comprise a moisturizer. In some cases, the benzoyl peroxide can dry out the region of the body to which the present drug compositions are applied. In these cases, it is helpful to include a moisturizer in the drug compositions to help the body retain the moisture by combating the drying effect of the benzoyl peroxide.

The moisturizer is preferably present in the present compositions at a concentration of about 2% to about 6% by weight of the total composition. In a particularly preferred embodiment, the moisturizer is present at a concentration of about 4% by weight of the total composition.

Preferred, non-limiting examples of such moisturizers useful in the present compositions include glycerin, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, octyldodecanol, dipropylene glycol, sorbitol, derivatives thereof, and mixtures thereof. In a preferred embodiment, the moisturizer included in the present compositions comprises glycerin.

Gelling Agent

The presently preferred compositions may further comprise a gelling agent as an essential component. This gelling agent can provide the present drug compositions with a matrix for forming a low viscosity gel network. The gelling agent can form a three-dimensional network in the dispersant (the water and surfactant system in the present drug compositions), allowing individual particles present in the gelling agent to be linked to one another more or less firmly via electrostatic interaction.

In a preferred embodiment, the gelling agent is present in the instant compositions in an amount of about 0.2% to about 0.4% by weight. In a particularly preferred embodiment, the drug compositions comprise about 0.25 to about 0.35% by weight of the gelling agent. In a still further particularly preferred embodiment, the drug compositions comprise about 0.3% by weight of the gelling agent.

The gelling agent can be any substance that provides the necessary three-dimensional network within the dispersant. Preferred, non-limiting examples of gelling agents useful in this regard include various cellulose agents, such as hydroxyethylcellulose, cellulose gum, methylcellulose, hydroxypropylcellulose, hydroxypropylmethylcellulose, xanthan gum, gum arabic, gum tragacanth, locust bean gum, guar gum, other cellulosic polymers, derivatives thereof, and mixtures thereof.

Other suitable gelling agents useful in the present compositions include sodium carbomer, carbomer, polyacrylic polymers, aqueous gelling agents, such as neutral, anionic, cationic polymers, carboxy vinyl polymers, such as carboxypolymethylene, derivatives thereof, and mixtures thereof. In a particularly preferred embodiment, the gelling agent is carbomer.

In this regard, a specific preferred gelling agent is a Carbopol® polymer (i.e. a polyacrylic polymer) such as is available from Noveon Inc., Cleveland, Ohio. Another particularly preferred gelling agent is a polyacrylic polymer, for example a copolymer of acrylic acid and a long chain alkyl methacrylate. This copolymer can be crosslinked with polyalkenyl ethers of polyalcohols, for example as with a Pemulen® polymer available from Noveon Inc., Cleveland, Ohio.

Dermatologically Acceptable Excipients

The preferred drug compositions discussed herein can additionally comprise at least one dermatologically acceptable excipient commonly known to those of ordinary skill in the art as useful in topical compositions. Preferred, non-limiting examples of dermatologically acceptable excipients useful in these drug compositions are those selected from the group consisting of preservatives, colorants or pigments, anti-oxidants, radical scavengers, emulsifiers, humectants, pH modifiers, chelating agents, derivatives thereof, and mixtures thereof.

Preservatives

The presently preferred drug compositions may optionally further contain at least one preservative. Preferred non-limiting examples of preservatives useful in this regard include propylene glycol, glycerol, butylene glycol, pentylene glycol, hexylene glycol, sorbitol, benzyl alcohol, ethanol, derivatives thereof, and mixtures thereof.

The preservative is preferably present in an amount of about 0.1% to about 2.5% by weight of the overall weight of the composition.

Anti-oxidants

The presently preferred drug compositions may optionally further contain at least one anti-oxidant. Preferred non-limiting examples of antioxidants useful in this regard include ascorbic acid, ascorbyl esters of fatty acids, magnesium ascorbyl phosphate, sodium ascorbyl phosphate, ascorbyl sorbate, tocopherol, tocopherol sorbate, tocopherol acetate, butylated hydroxy benzoic acid, thioglycolates, persulfate salts, 6-hydroxy-2,5,7,8-tetramethylchroman-2-carboxylic acid, lipoic acid, gallic acid, propyl gallate, uric acid, sorbic acid, lipoic acid, amines, N,N-diethylhydroxylamine, N-acetyl-L-cysteine, amino-guanidine, sulfhydryl compounds, glutathione, dihydroxy fumaric acid, lycine pidolate, arginine pilolate, nordihydroguaiaretic acid, bioflavonoids, curcumin, lysine, 1-methionine, proline, superoxide dismutase, silymarin, tea extracts, grape skin/seed extracts, melanin, rosemary extracts, derivatives thereof, and mixtures thereof.

Humectants

The presently preferred drug compositions may optionally further contain a humectant. Preferred, non-limiting examples of humectants useful in this regard include sorbitol, sorbitol syrup, E965 maltitol, maltitol, maltitol syrup, E1200 polydextrose, E1518 glyceryl triacetate, triacetin, glyceryl triacetate, 1,2,3-propanetriyl triacetate, 1,2,3-propanetriol triacetate, triacetylglycerol, E1520 propylene glycol, 1,2-propanediol, 1,2-dihydroxypropane, methylethylene glycol, propane-1,2-diol, E420 sorbitol, propylene glycol, polyethylene glycol (PEG) esters, PEG-20 stearate, PEG-40 stearate, PEG-150 stearate, PEG-150 distearate, PEG-100 stearate, laureth-12, ceteareth-20, laureth-23, glycereth-7, glycereth-12, glycereth-26, PEG-4, PEG-6, PEG-8, PEG-12, PEG-32, PEG-75, PEG-150, derivatives thereof, and mixtures thereof.

pH Modifiers

The presently preferred drug compositions may optionally further contain a pH modifier. Preferred non-limiting examples of pH modifiers useful in this regard include inorganic hydroxides, inorganic oxides, inorganic salts of weak acids, inorganic acids, organic acids, derivatives thereof, and mixtures thereof.

Preferred, non-limiting examples of inorganic hydroxides useful in this regard include ammonium hydroxide, alkali metal hydroxide, alkaline earth metal hydroxides, derivatives thereof, and mixtures thereof.

Preferred non-limiting examples of inorganic hydroxides useful herein include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred, non-limiting examples of inorganic oxides useful herein include magnesium oxide, calcium oxide, derivatives thereof, and mixtures thereof.

Preferred, non-limiting examples of inorganic salts of weak acids useful herein include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

Preferred, non-limiting examples of inorganic acids useful herein include hydrochloric acid, hydrofluoric acid, hydrobromic acid, nitric acid, nitrous acid, hydrocyanic acid, perchloric acid, chlorous acid, sulfurous acid, hypochlorous acid, phosphoric acid, acetic acid, sulfuric acid, derivatives thereof, and mixtures thereof.

Preferred, non-limiting examples of organic acids useful herein include lactic acid, citric acid, glutamic acid, methanoic acid, ethanoic acid, phenol, monochloroethanoic acid, dichloroethanoic acid, trichloroethanoic acid, butanoic acid, salicylic acid, glycolic acid, and mixtures thereof.

Further, mixtures of any of the above-mentioned pH modifiers are also contemplated as within the scope of the present compositions.

Chelating Agents

The presently preferred drug compositions may optionally further contain a chelating agent. Preferred non-limiting examples of chelating agents useful in this regard include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosponates, pharmaceutically acceptable salts thereof, derivatives thereof, and mixtures thereof.

In addition to those enumerated above, any other surfactant, moisturizer, gelling agent, preservative, colorant or pigment, antioxidant, radical scavenger, emulsifier, humectant, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein.

Well-known carriers used to formulate other topical therapeutic compositions for administration to humans will be useful in these compositions. Examples of such components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

Pads

The present topical drug delivery systems may also contain at least one pad to deliver the composition to a patient in need thereof. The pad of the delivery system allows for easy application of the drug compositions to the skin of a user. Accordingly, the pad is preferably made of a material in which the drug composition is capable of being absorbed.

In this regard, exemplary applicator pads useful in the present subject matter are made, by way of non-limiting example, from a plastic foam, a sponge, a woven or nonwoven natural or synthetic fiber or fabric, including gauze, felt, or cotton, or any other material capable of absorbing the present drug compositions. In a preferred embodiment, is made of synthetic or natural material and woven or non-woven material. In a particularly preferred embodiment, the pad the pad is made of a non-woven synthetic material, for example without limitation, BBA Fiberweb®. The BBA Fiberweb® pad contains 75% rayon and 25% polypropylene. It is a thermal bonded fabric made by BBA Nonwoven Division, Bethune, S.C. The pad is preferably disposable.

The pads useful herein can be composed of a single layer, or they can be formed of two or more layers. As will be apparent to a skilled artisan, for multiple layer pads, the various layers can be made of the same or different materials. The pads can also be of various forms or shapes, for example, with the substrate in a rectangular or washcloth-like shape, or alternatively, the substrate can be in the form of a glove, mitt or mitten. A wide variety of additional shapes are possible, such as oval, circular, etc. In a preferred embodiment, the pads are circular in shape.

Container

The delivery systems of the present subject matter may also include a container. If present, the container holds the at least one pad and the drug composition absorbed thereon.

The container may contain one or more pads and does not leak the composition from the pads. The container aids in preserving the pad(s) and drug composition once it is sealed. In one embodiment of the present subject matter, the container may be any material that packages the pad with the composition and does not degrade or leak the composition for a sufficient period of time, such as the shelf life of the product. In a preferred embodiment, the container comprises one or more sheets of plastic-lined foil material. The container is preferably fashioned from the sheets to hold one or more pads.

In one embodiment, two sheets, which are larger than the pad or the dimensions of the pad when folded, are placed in the following layers: a bottom sheet, the pad, and the top sheet over the pad. The sides of the sheets meet because the pad is smaller than the sheets and is placed in the center of the sheets. At the sides of the sheets, heat and pressure is applied which causes the plastic lining of both sheets to melt and seal together, enclosing the pad. One of ordinary skill in the art will understand that time, heat, and pressure will vary according to the type of material and/or process used.

If a plurality of pads is held within the container formed of a lined foil material, the container is preferably re-sealable in order to maintain the stability of the drug compositions absorbed onto the pads.

In an alternative embodiment, the container comprises a plastic or glass jar suitable for holding the pads and the drug composition. The plastic or glass jar is made of a suitable plastic or glass material that does not react with the drug composition, and may have an inner seal between the top of the jar and the lid of the jar. The inner seal helps maintain an inert atmosphere within the jar, thereby extending the shelf life of the drug compositions absorbed onto the pads stored in the jar.

When the container is a plastic jar, the plastic used to make the jar may comprise, without limitation, polyvinyl chloride, polyethylene, polypropylene, polyester, any other suitable plastic material, and mixtures thereof. Whether the jar is plastic or glass, it is preferred to tint the plastic or glass jar with a dye which may help to protect the drug compositions from harmful ultraviolet rays.

A skilled artisan will recognize that any suitable container can be used to hold the at least one pad and drug composition of the present subject matter. Preferably, the container is re-sealable and holds a plurality of pads.

### Methods of Treatment

Another preferred aspect of the present subject matter pertains to a method for treating acne comprising administering to a patient in need thereof a drug composition comprising

about 2.5-13% by weight benzoyl peroxide in a suspension comprising
about 80-90% by weight water, and
about 0.1-1.5% by weight of a surfactant system;
   wherein the drug composition is administered to the patient using a pad on which the drug composition is absorbed. In a particularly preferred aspect, the pad and the drug composition are stored in a container.

In a further embodiment, the present subject matter is drawn to a method for treating acne comprising administering to a patient in need thereof a drug composition comprising

about 2.5-13% by weight benzoyl peroxide in a suspension comprising
about 80-90% by weight water,
about 0.1-1.5% by weight of a surfactant system,
about 0.2-0.4% by weight of a gelling agent, and
about 2-6% by weight of a moisturizer;
   wherein said drug composition is administered to the patient using a pad on which said drug composition is absorbed. In a particularly preferred aspect of this embodiment, the pad and drug composition are stored in a container.

The present subject matter also contemplates the treatment of other skin disorders by applying the present drug compositions to the skin of a patient. Exemplary specific skin disorders, other than acne, treatable by the present compositions include but are not limited to impetigo, rosacea, atopic dermatitis,. secondary skin infections, seborrhea, skin lesions, and bacterial skin infections. In a preferred embodiment, the skin disorder or condition improves following treatment with the present compositions.

### Combination Therapy

In another preferred embodiment, the present preferred compositions may be used in combination with an additional pharmaceutical dosage form to enhance their effectiveness in treating dermatological disorders described herein. In this regard, the present preferred compositions may be administered as part of a regimen additionally including any other pharmaceutical and/or pharmaceutical dosage form known in the art as effective for the treatment of acne generally, or one of these disorders specifically.

In addition to the benzoyl peroxide, the present compositions may further contain other active ingredients readily known to those of skill in the art as useful in the topical treatment of acne. Exemplary additional active ingredients include, but are not limited to, macrolide antibiotics, bactericidal drugs, bacteriostatic drugs, cleansing agents, absorbents, anti-infective agents, anti-inflammatory agents, astringents (drying agents that precipitate protein and shrink and contract the skin), emollients (skin softeners), keratolytics (agents that soften, loosen, and facilitate exfoliation of the squamous cells of the epidermis), and mixtures thereof.

Exemplary macrolide antibiotics contemplated as optionally within the scope of the present subject matter include, but are not limited to, Azithromycin, Clarithromycin, Erythromycin, Lincomycin, and mixtures thereof. The macrolides are similar in structure and activity. All the macrolides are easily absorbed and all are primarily bacteriostatic and bind to the 50S subunit of the ribosome, thus inhibiting bacterial protein synthesis. These drugs are active against aerobic and anaerobic gram-positive cocci, with the exception of enterococci, and against gram-negative anaerobes and useful in combination with the present compositions.

Exemplary bactericidal drugs (i.e., they kill bacteria) contemplated as optionally within the scope of the present subject matter include, but are not limited to, penicillins, cephalosporins, vancomycin, aminoglycosides, quinolones, and polymyxins.

Exemplary bacteriostatic drugs (i.e., they slow bacterial growth) contemplated as optionally within the scope of the present subject matter include, but are not limited to, erythromycin, tetracyclines, chloramphenicol, clindamycin, lincomycin, clarithromycin, azithromycin, and sulfonamides. However, it is well know that some bactericidal drugs may be bacteriostatic against certain microorganisms and vice versa. These drugs are well known in the art and may be found, for example, in The Merck Manual of Diagnosis and Therapy, 13th edition, Section 13, Chapter 153 Anti-bacterial Drugs, 2001, incorporated herein by reference in its entirety.

Furthermore, the preferred formulations herein may be used with adjunct therapies and treatments, such as pre-washing with common soaps, and mild detergents. However, selection of such adjunct therapies is important when treating skin disorders such as acne since antibacterial soaps and abrasive soaps may increase irritation and make it difficult to use follicular drugs. Such follicular drugs may include topical antibiotics and antiseptics, as well as intralesional corticosteroids.

In superficial pustular acne, the topical drug compositions may be used in combination with one of the follicular drugs.

Sunlight therapy can be useful in combination with the present subject matter. Sunlight is known to cause mild dryness and slight scaling and is usually helpful. Since sunlight is not always available, some benefit may be obtained with a sunlamp.

Another combination therapy involves azelaic acid cream 20%, which has antiproliferative and antibacterial effects, and is known to be effective in comedonal or inflammatory acne.

An additional combination therapy contemplated herein is topical tretinoin (retinoic acid) in 0.025%, 0.05%, or 0.1% cream, 0.05% liquid, or 0.01% or 0.025% gel. Also, a new topical retinoid, Differinn brand adapalene 0.1% gel, Galderma Laboratories, San Antonio, Tex., was recently approved in the USA and may be useful since it may be slightly less irritating than topical tretinoin. Other retinoids which may be useful in combination therapy include Panretin®, containing alitretinoin, and Targretin®, containing bexarotene. Since retinoids must be applied carefully and at night to avoid excessive irritation, a regimen in combination with these drugs may be used over time to achieve results. For example, retinoid therapy may be initiated and then followed on with once a day treatment in accordance with the present subject matter. Exposure to sunlight when using retinoids and concurrent use of other drugs are restricted to prevent severe irritation. However, a back-to-back alternating regimen over a period of weeks or months time may be useful. With tretinoin or adapalene, acne may worsen at first; improvement usually requires ≧3 to 4 weeks.

Other topical drugs include OTC drugs, various sulfur-resorcinol combinations, and oral antibiotics may also be helpful in combination with the present subject matter when treating acne.

Similarly, an anti-acne agent other than those specified herein, or an additional topical pharmaceutically active agent, can be added to the present preferred compositions to enhance their effectiveness. Accordingly, this additional agent or additional pharmaceutical dosage form can be applied to a patient either directly or indirectly, and concomitantly or sequentially, with the preferred compositions described herein.

Further, in this embodiment, simultaneous application of the preferred drug compositions with administration of an oral antibiotic may provide synergistic beneficial effects in the treatment of acne. Such synergistic effects may be enhanced by the simultaneous topical application of the preferred drug compositions herein with oral administration of the antibiotics.

### Methods of Production

The present subject matter is also directed to a method for making the preferred drug compositions and delivery systems. A preferred method for making the drug compositions and delivery systems comprises:
1) Adding water to a suitable vessel and heating the water to about 60-80° C.;
2) Adding a non-ionic surfactant and an anionic surfactant to the water vessel;
3) Cooling the above mixture to a temperature of about 20-25° C. while maintaining mixing of the mixture;
4) In a separate vessel, adding this mixture with water and benzoyl peroxide and milling the resultant mixture to reduce particle size to no greater than 60 microns;
5) Adding water to the mixture from step 4) and mixing for at least 30 minutes; and
6) Absorbing the mixture from step 5) onto and into suitable pads.

In a preferred embodiment, a moisturizer and a preservative are added to the water vessel in step 2) along with the non-ionic surfactant and the anionic surfactant. In a further preferred embodiment, the non-ionic surfactant, moisturizer, preservative, and anionic surfactant are successively added to the water vessel

In another preferred embodiment, a gelling agent is added to the water vessel prior to the step 2) above. According to this further step, the gelling agent/water mixture is mixed until the gelling agent is suitably hydrated. In a particularly preferred embodiment, the gelling agent/water mixture will be mixed for at least about 15 minutes.

In still another preferred embodiment, a 1-5% sodium hydroxide solution containing purified water and sodium hydroxide is prepared in a separate container by mixing the solution with a spatula until the sodium hydroxide is dissolved. This solution can then be added to the mixture of step 4) above and mixed for at least 10 minutes.

In another alternative preferred embodiment, the pH of the mixture from step 5) above can be adjusted to at least a pH of at least 5, by adding a sodium hydroxide solution to the mixture from step 5) above.

### Dosage

To be effective, the route of administration for the compositions used in the present methods and pharmaceutical compositions must readily affect the target areas. In particular, acne is known to affect the face, neck, back, ears, and scalp. Moreover, it will be understood that a useful dosage of benzoyl peroxide can be administered in a single or multiple dosage units to provide the desired therapeutic effect.

The preferred drug compositions may be given in a single or multiple doses daily. In a preferred embodiment, the pharmaceutical compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of benzoyl peroxide in the patient's body; the age, body weight, general health, sex and diet of the patient; the time of administration; the rate of excretion; drug combination; the severity of the acne being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, Remington's Pharmaceutical Sciences, 18th ed. (1990, Mack Publishing Co., Easton, Pa. 18042), pp. 1435-1712, the disclosure of which is hereby incorporated by reference in its entirety. Such formulations may influence the physical state, stability, rate of in vivo release, and rate of in vivo clearance of the therapeutic agents.

### EXAMPLES

The following examples are illustrative of preferred compositions and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a preferred delivery system of the present subject matter. The pads of the delivery system have absorbed thereon a 4% benzoyl peroxide drug composition comprising:

| | % W/W |
|---|---|
| Purified Water | 88.71 |
| Carbomer 940, NF | 0.50 |
| Poloxamer 182 | 0.20 |
| Benzoyl peroxide (75%), USP | 6.13 |
| Glycerin, USP | 4.00 |
| Methylparaben, NF | 0.04 |
| Disodium lauryl sulfosuccinate | 0.04 |
| Silicon dioxide, NF | 0.25 |
| Edetate disodium | 0.10 |
| Sodium Hydroxide | 0.03 |
| | 100.0% |

Preparation of the drug composition and delivery system:
1) Adding 829.6 kg of water to a suitable vessel and heating the water to about 70° C.;
2) Adding 5.0 kg of Carbomer 940 to the water and mixing the Carbomer/water mixture for about 15 minutes, or until the Carbomer is suitably hydrated;
3) Adding successively 2.0 kg of poloxamer 182, 40.0 kg of glycerin, 0.4 kg of methylparaben, 2.5 kg of silicon dioxide, 0.4 kg of disodium lauryl sulfosuccinate, and 1.0 kg of edentate disodium to the carbomer mixture;
4) Cooling the above mixture to a temperature of about 20-25° C. while maintaining mixing of the mixture;
5) In a further vessel, adding 3.0 kg of water and 0.3 kg of sodium hydroxide;
6) Adding the sodium hydroxide solution to the further mixture from step 4) and mixing for 10 minutes;
7) In a separate vessel, adding 125.0 kg of the mixture from step 6) with 70.8. kg of water and 46.0 kg of benzoyl peroxide and milling the resultant mixture to reduce particle size to no greater than 60 microns;
8) Adding 40.0 kg of water to the mixture from step 7) and mixing for 30 minutes;
9) Adjusting the pH of the mixture to at least a pH of 5.5 by adding sodium hydroxide solution to the mixture from step 8);
10) Adding the remaining water and mixing for 5 minutes;
11) Absorbing the mixture from step 10) onto and into suitable pads.

### Example 2

The following example illustrates the preparation of a preferred delivery system of the present subject matter. The pads of the delivery system have absorbed thereon a 4% benzoyl peroxide drug composition comprising:

| | % W/W |
|---|---|
| Purified Water | 88.72 |
| Carbomer 940, NF | 0.30 |
| Poloxamer 182 | 0.20 |
| Benzoyl peroxide (75%), USP | 6.20 |
| Glycerin, USP | 4.00 |
| Methylparaben, NF | 0.10 |
| Disodium lauryl sulfosuccinate | 0.10 |
| Hydrated Silica | 0.25 |
| Disodium EDTA | 0.10 |
| Sodium Hydroxide | 0.03 |
| | 100.0% |

This drug composition was prepared according to the procedure set forth above with respect to Example 1. This drug composition was then absorbed onto and into suitable pads to produce the present inventive delivery system.

### Example 3

The following example illustrates the preparation of a preferred delivery system of the present subject matter. The pads of the delivery system have absorbed thereon a 4% benzoyl peroxide drug composition comprising:

| | % W/W |
|---|---|
| Purified Water | 88.85 |
| Carbomer 940, NF | 0.30 |
| Poloxamer 182 | 0.20 |
| Benzoyl peroxide (75%), USP | 6.13 |
| Glycerin, USP | 4.00 |
| Methylparaben, NF | 0.10 |
| Disodium lauryl sulfosuccinate | 0.04 |
| Silicon dioxide, NF | 0.25 |
| Edetate disodium | 0.10 |
| Sodium Hydroxide | 0.03 |
| | 100.0% |

This drug composition was prepared according to the procedure set forth above with respect to Example 1. This drug composition was then absorbed onto and into suitable pads to produce the present inventive delivery system.

### Example 4

The following example illustrates the preparation of a preferred delivery system of the present subject matter. The pads of the delivery system have absorbed thereon a 8% benzoyl peroxide drug composition comprising:

| | % W/W |
|---|---|
| Purified Water | 82.54 |
| Carbomer 940, NF | 0.50 |
| Poloxamer 182 | 0.20 |
| Benzoyl peroxide (75%), USP | 12.30 |
| Glycerin, USP | 4.00 |
| Methylparaben, NF | 0.04 |
| Disodium lauryl sulfosuccinate | 0.04 |
| Silicon dioxide | 0.25 |
| Disodium EDTA | 0.10 |
| Sodium Hydroxide | 0.03 |
| | 100.0% |

This drug composition was prepared according to the procedure set forth above with respect to Example 1. This drug composition was then absorbed onto and into suitable pads to produce the present inventive delivery system.

### Example 5

A patient is suffering from acne. The drug composition of Example 1 is topically administered to the patient by way of drug composition-laden pads. It would be expected that the patient would improve his/her condition or recover.

### Example 6

A patient is suffering from acne. The drug composition of Example 2 is topically administered to the patient by way of drug composition-laden pads. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A topical drug delivery system comprising:
at least one pad;
a container; and
a drug composition comprising about 2.5-13.0% by weight benzoyl peroxide in a suspension comprising about 80.0-90.0% by weight water and about 0.10-1.50% by weight of a surfactant system;
wherein said drug composition is absorbed onto said pad, and said container holds said pad with said drug composition.

2. The topical drug deliver system according to claim 1 wherein said surfactant system comprises about 50-95% by weight of at least one nonionic surfactant and about 5-50% by weight of at least one anionic surfactant.

3. The topical drug delivery system according to claim 2 wherein said at least one nonionic surfactant is selected from the group consisting of alkanolamides, amine oxides, esterified carboxylic acids, ethoxylated alcohols, poloxamers, and mixtures thereof.

4. The topical drug delivery system according to claim 3 wherein said at least one nonionic surfactant is a poloxamer.

5. The topical drug delivery system according to claim 2 wherein said at least one anionic surfactant is selected from the group consisting of carboxylates, amino acid derivatives, alkyl sulphates, alkyl ether sulfates, sulphonates, isethionates, taurates, sulfosuccinates, alkyl sulfoacetates, phosphates, alkyl phosphates, and mixtures thereof.

6. The topical drug delivery system according to claim 5 wherein said at least one anionic surfactant is disodium lauryl sulfosuccinate.

7. The topical drug delivery system according to claim 1 wherein said container holds a single pad.

8. The topical drug delivery system according to claim 1 wherein said container holds a plurality of pads.

9. A topical drug delivery system comprising:
at least one pad; and
a drug composition comprising about 2.5-13.0% by weight benzoyl peroxide in a suspension comprising about 80.0-90.0% by weight water and about 0.10-1.50% by weight of a surfactant system;
wherein said drug composition is absorbed onto said pad.

10. The topical drug deliver system according to claim 9 wherein said surfactant system comprises about 50-95% by weight of at least one nonionic surfactant and about 5-50% by weight of at least one anionic surfactant.

11. The topical drug delivery system according to claim 9 further comprising a container, wherein said container holds a single pad.

12. The topical drug delivery system according to claim 9 further comprising a container, wherein said container holds a plurality of pads.

13. A topical drug delivery system comprising:
at least one pad;
a container; and
a drug composition comprising about 2.5-13.0% by weight benzoyl peroxide in a suspension comprising about 80.0-90.0% by weight water, about 0.10-1.50% by weight of a surfactant system, about 0.20-0.40% by weight of a gelling agent, and about 2.0-6.0% by weight of a moisturizer;
wherein said drug composition is absorbed onto said pad, and said container holds said pad with said drug composition.

14. The topical drug deliver system according to claim 13 wherein said surfactant system comprises about 50-95% by weight of at least one nonionic surfactant and about 5-50% by weight of at least one anionic surfactant.

15. The topical drug delivery system according to claim 13 wherein said container holds a single pad.

16. The topical drug delivery system according to claim 13 wherein said container holds a plurality of pads.

17. A method for treating acne comprising administering to a patient in need thereof a drug composition comprising about 2.5-13.0% by weight benzoyl peroxide in a suspension comprising about 80.0-90.0% by weight water and about 0.10-1.50% by weight of a surfactant system; wherein said drug composition is administered using a pad on which said drug composition is absorbed.

18. The method according to claim 17 wherein said surfactant system comprises about 50-95% by weight of at least one nonionic surfactant and about 5-50% by weight of at least one anionic surfactant.

19. The method according to claim 17 wherein, prior to use, said pad is stored in a container.

20. The method according to claim 19 wherein said container holds a plurality of pads.

21. The method according to claim 17 wherein said drug composition is administered in conjunction with another therapeutic composition effected for treating acne.

22. The method according to claim 21 wherein said another therapeutic composition is administered either concomitantly or sequentially with said drug composition.

23. A method for treating acne comprising administering to a patient in need thereof a drug composition comprising about 2.5-13.0% by weight benzoyl peroxide in a suspension comprising about 80.0-90.0% by weight water, about 0.10-1.50% by weight of a surfactant system, about 0.20-0.40% by weight of a gelling agent, and about 2.0-6.0% by weight of a moisturizer; wherein said drug composition is administered using a pad on which said drug composition is absorbed.

24. The method according to claim 23 wherein said surfactant system comprises about 50-95% by weight of at least one nonionic surfactant and about 5-50% by weight of at least one anionic surfactant.

25. The method according to claim 23 wherein, prior to use, said pad is stored in a container.

26. The method according to claim 25 wherein said container holds a plurality of pads.

27. The method according to claim 23 wherein said drug composition is administered in conjunction with another therapeutic composition effective for treating acne.

28. The method according to claim 27 wherein said another therapeutic composition is administered either concomitantly or sequentially with said drug composition.
